(19) United States
(12) Patent Application Publication
(10) Pub. No.: US 2005/0152957 A1
(43) Pub. Date: Jul. 14, 2005

(54) Compositions and delivery systems for administration of a local anesthetic agent

(75) Inventors: Gary W. Cleary, Los Altos Hills, CA (US); Sri Mudumba, Union City, CA (US); Shoreh Parandoosh, Menlo Park, CA (US); Colin J. Cleary, San Mateo, CA (US); Raj Birudaraj, Belmont, CA (US); Pathamar Park, Moss Beach, CA (US)

Correspondence Address: REED INTELLECTUAL PROPERTY LAW GROUP, 1400 PAGE MILL ROAD, PALO ALTO, CA, 94304-1124, US

(73) Assignee: CORIUM INTERNATIONAL, Redwood City, CA (US)

(21) Appl. No.: 11/077,593

(22) Filed: Mar. 10, 2005

Related U.S. Application Data
(63) Continuation of application No. 10/141,496, filed on May 7, 2002, now abandoned.
(60) Provisional application No. 60/289,403, filed on May 7, 2001.

(51) Int. Cl. A61K 31/24; A61L 15/16

(52) U.S. Cl. 424/448; 514/536

(57) ABSTRACT

A pharmaceutical composition is provided for topical administration of a local anesthetic agent. The composition comprises (a) a therapeutically effective amount of a local anesthetic agent and (b) a pharmaceutically acceptable, nonliposomal carrier comprised of a monohydric alcohol, a penetration enhancer, and polymer, which may be a hydrophilic polymer, a hydrophobic polymer or a combination thereof. The composition can be in the form of a gel, or it may form a film following application to a patient's body surface and evaporation of the monohydric alcohol. The composition provides rapid onset of local anesthesia as well as penetration of the active agent into the skin. Methods and drug delivery systems for administration of local anesthetic agents are also provided.

### CROSS-REFERENCE TO RELATED APPLICATION

This application claims priority to U.S. Ser. No. 10/141,496, filed May 7, 2002, which claims priority under 35 U.S.C. §119(e)(1) to U.S. Provisional Application Ser. No. 60/289,403, filed May 7, 2001; the disclosures of both are incorporated herein by reference.

### TECHNICAL FIELD

The invention relates generally to pharmaceutical compositions and drug delivery systems, and more particularly relates to pharmaceutical compositions and drug delivery systems for administration of an anesthetic agent, particularly a local anesthetic agent. In addition, the invention relates to methods for administering local anesthetic agents to patients using the aforementioned pharmaceutical compositions and drug delivery systems.

### BACKGROUND

Anesthetic agents are widely used in the management of pain. Pharmacologically, most anesthetic agents reversibly block the conduction of pulses along nerve axons and other excitable membranes. Clinically, local administration of an anesthetic agent results in selective anesthesia, i.e., anesthesia limited to the area near the location of administration, and only for a limited quantity of time, e.g., on the order of four hours or less. Thus, local anesthetic agents derive their clinical usefulness and popularity by selectively, and reversibly, blocking noxious nerve impulses.

Administration of a local anesthetic agent can be used for a variety of purposes. For example, local anesthetic agents can be used to treat noxious stimuli caused by irritation from an environmental toxin, e.g., a toxic resin of a poisonous plant. Local anesthetic agents have also been used to treat the pain associated with wounds or tissue damage. Often, local anesthetic agents are administered to an individual undergoing a medical, dental or cosmetic procedure to treat the pain associated with such procedures. For these uses, the local anesthetic agent is often administered prior to undergoing the procedure so that any pain associated therewith is ameliorated or eliminated.

Cocaine, one of the first local anesthetic agents, possesses strong anesthetic properties and was initially used as an ophthalmic anesthetic agent. Although very effective in producing local anesthesia, cocaine's highly addictive nature was soon discovered and less-addictive alternatives were sought. Researchers synthesized new local anesthetic agents based on the chemical structure of cocaine. Currently, many local anesthetic agents are available that are non-addicting.

One drawback of local administration is that needles are often used to inject the drug, thereby causing pain to the patient. Often, the mere presence of a needle triggers anxiety, fear and discomfort. Gupta et al. (1996) J. Am. Acad. Dermatol. 35(3):419-423. This is particularly true for children and patients with a low pain threshold, making treatment of these individuals difficult. Ideally, administration of a local anesthetic agent should not be painful.

One approach for minimizing the pain associated with local administration of an anesthetic agent is the topical administration of the agent via a suitable delivery system, e.g., patch, or composition, e.g., cream, gel or ointment. Unlike direct injection, topical administration does not require the use of needles. In addition, topical administration does not require the expertise of a nurse or other skilled caregiver, thereby allowing for the ease and convenience of self-administration.

Topical administration, however, is not without drawbacks. For example, topical delivery of an anesthetic agent typically involves a delay prior to the onset of anesthesia. The delay is attributable to the time it takes for the anesthetic agent to reach the targeted tissue area. For example, EMLA® brand of lidocaine and prilocaine cream (AstraZeneca, Westborough, Mass.) is commonly applied sixty minutes before initiating a potentially painful dermal procedure. Riendeau et al. (1999) Reg. Anesth. Pain Med. 24(2):165-169. Such a delay can be inconvenient and may force some patients undergoing certain medical or cosmetic procedures to wait before the procedure can begin. Some have even suggested that clinics reorganize their procedures in order to accommodate such long waiting periods. Robieux et al (1990) Can. J Hosp. Pharm. 43(5):235-236. Clearly, it would be advantageous to have a topical formulation or device that would provide local anesthesia more quickly.

Another drawback commonly associated with topical administration of active agents is that the active agent may not penetrate into the deeper layers of the skin. This may be particularly problematic with a local anesthetic agent. For example, the cause of the pain may reside beneath the upper layers of the skin, beyond the reach of many conventional topical pain-relieving compositions. Thus, compositions and devices are desired that provide enhanced penetration of a topically administered local anesthetic agent.

Some have suggested using permeation enhancers to address the delayed onset of anesthesia and/or insufficient penetration of the active agent. U.S. Pat. No. 5,912,271 to Brodin et al. describes a composition comprising an anesthetic agent, a triacylglycerol, and one or more polar lipids. The described composition is stated to provide anesthesia in as little as fifteen minutes following application to a body surface. However, the triacylglycerol must be very pure and free from other glycerides. Such purity requires additional processing and testing steps, thereby increasing the complexity of the formulation.

U.S. Pat. No. 4,557,934 to Cooper describes the use of 1-dodecylazacycloheptan-2-one (commercially referred to as Azone®) as a penetration enhancer for local anesthetic agents and other drugs. Although this and other permeation enhancers may provide some measure of increased penetration, the need still exists for compositions and delivery systems for administering a local anesthetic agent that will have a relatively fast onset of action and be able to more deeply penetrate the skin. The present invention satisfies this and other needs in the art.

### SUMMARY OF THE INVENTION

Accordingly, it is a primary object of the invention to provide a pharmaceutical composition comprising a therapeutically effective amount of a local anesthetic agent in a pharmaceutically acceptable, nonliposomal carrier, wherein the nonliposomal carrier comprises a monohydric alcohol, a penetration enhancer, and a polymer selected from the group consisting of hydrophilic polymers, hydrophobic polymers and combinations thereof, wherein the local anesthetic activity is provided within about thirty minutes of application of the composition to a patient's body surface.

It is a further object of the invention to provide such a composition wherein the local anesthetic agent is selected from the group consisting of tetracaine, lidocaine, prilocaine, benzocaine, and combinations thereof.

It is still a further object of the invention to provide such a composition wherein the composition forms a water-soluble, water-insoluble, or water-resistant film upon application of the composition to a body surface of a patient.

It is a further object of the invention to provide such a composition in the form of a hydrophobic or hydrophilic gel.

It is a further object of the invention to provide a pharmaceutical composition comprising (a) a therapeutically effective amount of a local anesthetic agent and (b) a pharmaceutically acceptable, nonliposomal carrier comprised of a volatile monohydric alcohol, an effective enhancing amount of a penetration enhancer, and a polymer selected from the group consisting of hydrophilic polymers, hydrophobic polymers and combinations thereof, wherein the composition forms a film following application to a body surface of a patient and evaporation of the monohydric alcohol.

It is a further object of the invention to provide a drug delivery system for topical administration of a local anesthetic agent.

It is yet another object of the invention to provide a drug delivery system for mucosal administration of a local anesthetic agent.

A still further object of the invention is to provide a method for administering a local anesthetic agent to a patient by topically applying to the patient's body surface a pharmaceutical composition or drug delivery system as provided herein.

Additional objects, advantages and novel features of the invention will be set forth in the description that follows, and in part will become apparent to those skilled in the art upon examination of the following, or may be learned by practice of the invention.

In one embodiment, a pharmaceutical composition is provided comprising a therapeutically effective amount of a local anesthetic agent and a pharmaceutically acceptable, nonliposomal carrier that comprises a monohydric alcohol, an effective enhancing amount of a penetration enhancer, and a polymer selected from the group consisting of hydrophilic polymers, hydrophobic polymers and combinations thereof, wherein the carrier assists in providing local anesthetic activity within about thirty minutes of application of the composition to a patient's body surface. The local anesthetic agent is preferably blended with the carrier to form a consistent and homogenous admixture.

Depending on the polymer used and the relative amounts of the components in the nonliposomal carrier, the compositions can advantageously take one of several forms. For example, when a relatively large amount of a monohydric alcohol is present in the nonliposomal carrier, e.g., in the range of about 40 wt. % to about 90 wt. % based on the total weight of the composition, the composition forms a film following application to a body surface and concomitant and/or subsequent evaporation of the alcohol. Furthermore, depending on the polymer used, the film can be water soluble, water insoluble, or water resistant in nature. When a relatively smaller quantity of the monohydric alcohol is incorporated into the composition, e.g., less than about 40 wt. %, the composition forms a gel. Depending on the polymer used, the gel may be hydrophobic or hydrophilic.

In another embodiment, a drug delivery system is provided for topical administration of a local anesthetic agent. The system is a device in the form of a laminated composite having a drug reservoir layer containing a pharmaceutical composition and, optionally, an upper backing layer laminated to the drug reservoir layer. The pharmaceutical composition includes (i) a therapeutically effective amount of a local anesthetic agent, (ii) a monohydric alcohol, and (iii) an effective enhancing amount of a penetration enhancer. The backing layer, if present, serves as the outer surface of the device following application to a patient's body surface.

In still another embodiment, a drug delivery system is provided for mucosal administration of a local anesthetic agent. The system includes a drug reservoir layer that is water soluble; this is advantageous for buccal (or other transmucosal) drug delivery, wherein gradual and complete hydrolysis of the device in situ is desired. In this embodiment, the backing layer is absent, although the system may include a hydrophobic layer that serves as the outer surface of the device during use.

In yet another embodiment, a method is provided administering a local anesthetic agent to a patient. The method comprises applying a pharmaceutical composition or drug delivery system as provided herein to a localized region of the patient's body surface, e.g., the skin or mucosa.

Following application to a body surface, the compositions and delivery systems described herein provide immediate anesthesia to the desired tissues, i.e., local anesthesia occurs within about thirty minutes, preferably within about ten minutes, of application. In addition, the local anesthetic effect penetrates more deeply relative to conventional local anesthetic compositions and delivery systems. Finally, the anesthetic effect is generally prolonged relative to that obtained with conventional local anesthetic compositions and delivery systems, e.g., lasting at least 4 to 6 hours.

### BRIEF DESCRIPTION OF THE DRAWINGS

FIG. 1. is a graph comparing the in vivo anesthesia achieved with an anesthetic gel of the invention and with the commercially available ELAMAX® brand of topical anesthetic cream, as described in Example 1.

FIG. 2. is a graph comparing the in vivo anesthesia achieved with an anesthetic gel of the invention and with the commercially available EMLA® brand of cream, as described in Example 2.

FIG. 3. is a graph comparing the in vivo anesthesia achieved with an anesthetic gel of the invention and with the commercially available APETOP® brand of topical anesthetic cream as described in Example 3.

### DETAILED DESCRIPTION OF THE INVENTION

I. Definitions and Nomenclature

Before describing the present invention in detail, it is to be understood that unless otherwise indicated this invention is not limited to specific local anesthetic agents, monohydric alcohols, penetration enhancers, polymers, or the like, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a local anesthetic agent” includes a single local anesthetic agent as well as two or more local anesthetic agents, reference to “a polymer” includes a single polymer as well as combinations and mixtures of two or more polymers, and the like.

In describing and claiming the present invention, the following terminology will be used in accordance with the definitions set forth below.

The terms “active agent,” “drug” and “pharmacologically” are used interchangeably herein to refer to a chemical material or compound that, when administered to a patient (human or animal) induces a desired pharmacologic effect. Included are derivatives and analogs of those compounds or classes of compounds specifically mentioned that also induce the desired pharmacologic effect. Unless the context clearly dictates otherwise, the active agents referred to herein are local anesthetic agents.

The term “crosslinked” refers to a composition containing intramolecular and/or intermolecular crosslinks, whether arising through covalent or noncovalent bonding. “Noncovalent” bonding includes both hydrogen bonding and electrostatic (ionic) bonding.

The term “polymer” includes linear and branched polymer structures, and also encompasses crosslinked polymers as well as copolymers (which may or may not be crosslinked), thus including block copolymers, alternating copolymers, random copolymers, and the like. Those compounds referred to herein as “oligomers” are polymers having a molecular weight below about 1000 Da, preferably below about 800 Da.

The term “hydrogel” is used in the conventional sense to refer to water-swellable polymeric matrices that can absorb a substantial amount of water to form elastic gels, wherein “matrices” are three-dimensional networks of macromolecules held together by covalent or noncovalent crosslinks. Upon placement in an aqueous environment, dry hydrogels swell to the extent allowed by the degree of cross-linking.

The term “topical administration” is used in its conventional sense to mean application of an active agent to the skin or mucosa to achieve a local effect, as in, for example, topical drug administration in the prevention or treatment of pain. Topical administration herein may include transdermal delivery as well as transmucosal delivery, wherein the active agent passes through the skin or mucosal tissue and ultimately enters a patient's bloodstream.

The term “body surface” is used to refer to skin or mucosal tissue, including the interior surface of body cavities that have a mucosal lining. Thus, the term “body surface” contemplates the skin surface, the surface of a wound, the mucosa of the oral cavity, the surfaces of the vagina, and so forth. The term “skin” should be interpreted as including “mucosal tissue” and vice versa, unless the context clearly indicates otherwise.

By a “pharmaceutically acceptable carrier” is meant a material that is suitable for transdermal drug administration to an individual along with an active agent without causing any undesirable biological effects or interacting in a deleterious manner with any of the other components of the pharmaceutical formulation in which it is contained.

Similarly, a “pharmacologically acceptable” salt, ester, isomer or other derivative of an active agent as provided herein is a salt, ester, solvate, isomer or other derivative that is not biologically or otherwise undesirable.

By the terms “effective amount” and “therapeutically effective amount” of an active agent as provided herein is meant a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective amount” in any individual case may be determined by one of ordinary skill in the art using only routine experimentation.

The terms “treating” and “treatment” as used herein refer to the reduction in severity and/or frequency of symptoms and/or their underlying cause, and improvement or remediation of damage. Thus, for example, the present method of “treating” pain, as the term “treating” is used herein, encompasses both the prevention and initiation of a sensation of pain in the patient as well as the treatment of a patient experiencing pain.

The terms “condition,” “disease” and “disorder” are used interchangeably herein as referring to a physiological state that can be prevented or treated by administration of a composition or drug delivery device as described herein.

The term “patient” refers to a mammalian individual afflicted with or prone to a condition, disease or disorder as specified herein, and includes both humans and animals.

II. Anesthetic Compositions

A. Active Agents:

The active agent in the pharmaceutical compositions and drug delivery systems is a local anesthetic agent. Structurally, most local anesthetic agents contain a lipophilic group, e.g., an aromatic ring, a hydrocarbyl linking group (often having an amide or ester functionality), and an ionizable group, e.g., a tertiary amine. The invention, however, is not limited with respect to the molecular structure of the active agent.

Local anesthetic agents that can be administered using the compositions and drug delivery systems of the invention include, without limitation, acetamidoeugenol, alfadolone acetate, alfaxalone, amucaine, amolanone, amylocaine, benoxinate, benzocaine, betoxycaine, biphenamine, bupivacaine, burethamine, butacaine, butaben, butanilicaine, buthalital, butoxycaine, carticaine, 2-chloroprocaine, cocaethylene, cocaine, cyclomethycaine, dibucaine, dimethisoquin, dimethocaine, diperadon, dyclonine, ecgonidine, ecgonine, ethyl aminobenzoate, ethyl chloride, etidocaine, etoxadrol, β-eucaine, euprocin, fenalcomine, fomocaine, hexobarbital, hexylcaine, hydroxydione, hydroxyprocaine, hydroxytetracaine, isobutyl p-aminobenzoate, kentamine, leucinocaine mesylate, levoxadrol, lidocaine, mepivacaine, meprylcaine, metabutoxycaine, methohexital, methyl chloride, midazolam, myrtecaine, naepaine, octacaine, orthocaine, oxethazaine, parethoxycaine, phenacaine, phencyclidine, phenol, piperocaine, piridocaine, polidocanol, pramoxine, prilocaine, procaine, propanidid, propanocaine, proparacaine, propipocaine, propofol, propoxycaine, pseudococaine, pyrrocaine, risocaine, salicyl alcohol, tetracaine, thialbarbital, thimylal, thiobutabarbital, thiopental, tolycaine, trimecaine, zolamine, and combinations thereof.

Preferred local anesthetic agents are tetracaine, lidocaine, prilocaine, benzocaine, and combinations thereof, with tetracaine and lidocaine most preferred.

The amount of the local anesthetic agent contained in the compositions and drug delivery systems herein is a therapeutically effective amount. The therapeutically effective amount will generally although not necessarily be in the range of about 0.1 wt. % to about 50 wt. %, more preferably about 0.1 wt. % to about 30 wt. %, and most preferably about 0.1 wt. % to about 10 wt. % based on the total weight of the composition.

The compositions and systems described herein may include one or more additional active agents. Although any additional active agent suitable for topical or transdermal administration may be used, preferred additional active agents are as follows:

Bacteriostatic and bactericidal agents: Suitable bacteriostatic and bactericidal agents include, by way of example: halogen compounds such as iodine, iodopovidone complexes (i.e., complexes of PVP and iodine, also referred to as “povidine” and available under the tradename Betadine® from Purdue Frederick), iodide salts, chloramine, chlorohexidine, and sodium hypochlorite; silver and silver-containing compounds such as sulfadiazine, silver protein acetyltannate, silver nitrate, silver acetate, silver lactate, silver sulfate and silver chloride; organotin compounds such as tri-n-butyltin benzoate; zinc and zinc salts; oxidants, such as hydrogen peroxide and potassium permanganate; aryl mercury compounds, such as phenylmercury borate or merbromin; alkyl mercury compounds, such as thiomersal; phenols, such as thymol, o-phenyl phenol, 2-benzyl-4-chlorophenol, hexachlorophen and hexylresorcinol; and organic nitrogen compounds such as 8-hydroxyquinoline, chlorquinaldol, clioquinol, ethacridine, hexetidine, chlorhexedine, and ambazone.

Antibiotic agents: Suitable antibiotic agents include, but are not limited to, antibiotics of the lincomycin family (referring to a class of antibiotic agents originally recovered from streptomyces lincolnensis), antibiotics of the tetracycline family (referring to a class of antibiotic agents originally recovered from streptomyces aureofaciens), and sulfur-based antibiotics, i.e., sulfonamides. Exemplary antibiotics of the lincomycin family include I incomycin itself (6,8 dideoxy-6-[[(1-methyl-4-propyl-2-pyrrol idinyl)-carbonyl]amino]-1-thio-L-threo-α-D-galacto-octopyranoside), clindamycin, the 7-deoxy, 7-chloro derivative of lincomycin (i.e., 7-chloro-6,7,8-trideoxy-6-[[(1-methyl-4-propyl-2-pyrrol idinyl)carbonyl]amino]-1-thio-L-threo-α-D-galacto-octopyranoside), related compounds as described, for example, in U.S. Pat. Nos. 3,475,407, 3,509,127, 3,544,551 and 3,513,155, and pharmacologically acceptable salts and esters thereof. Exemplary antibiotics of the tetracycline family include tetracycline itself 4-(dimethylamino)-1,4,4α,5,5α,6,11,12α-octahydro-3,6,12,12α-pentahydroxy-6-methyl-1,11-dioxo-2-naphthacenecarboxamide), chlortetracycline, oxytetracycline, tetracycline, demeclocycline, rolitetracycline, methacycline and doxycycline and their pharmaceutically acceptable salts and esters, particularly acid addition salts such as the hydrochloride salt. Exemplary sulfur-based antibiotics include, but are not limited to, the sulfonamides sulfacetamide, sulfabenzamide, sulfadiazine, sulfadoxine, sulfamerazine, sulfamethazine, sulfamethizole, sulfamethoxazole, and pharmacologically acceptable salts and esters thereof, e.g., sulfacetamide sodium.

Topical Vasodilators: Such compounds are useful for increasing blood flow in the dermis, and preferred topical vasodilators are those known as rubefacients or counterirritants. Rubefacient agents include nicotinic acid, nicotinates such as methyl, ethyl, butoxyethyl, phenethyl and thurfyl nicotinate, as well as the essential oils such as mustard, turpentine, cajuput and capsicum oil, and components thereof. Particular preferred such compounds include, but are not limited to, methyl nicotinate, nicotinic acid, nonivamide, and capsaicin.

Proteolytic enzymes: Proteolytic enzymes include, for example, pepsin, trypsin, collagenase, chymotrypsin, elastase, carboxypeptidase, aminopeptidase, and the like.

Peptide, proteins, and amino acids: Suitable peptides and proteins are tissue-healing enhancing agents (also referred to in the art as “tissue regenerative agents”) such as collagen, glycosaminoglycans (e.g., hyaluronic acid, heparin, heparin sulfate, chondroitin sulfate, etc.), proteoglycans (e.g., versican, biglycan) substrate adhesion molecules (e.g., fibronectin, vitronectin, laminin), polypeptide growth factors (e.g., platelet-derived growth factor, a fibroblast growth factor, a transforming growth factor, an insulin-like growth factor, etc.), and other peptides such as fibronectin, vitronectin, osteopontin, and thrombospondin, all of which contain the tripeptide sequence RGD (arginine-glycine-aspartic acid), a sequence generally associated with adhesive proteins and necessary for interaction with cell surface receptors.

Salts, esters, amides, and derivatives of the active agent(s) may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, acid addition salts are prepared from the free base (e.g., compounds having a neutral —NH₂ or cyclic amine group) using conventional means, involving reaction with a suitable acid. Typically, the base form of an active agent is dissolved in a polar organic solvent such as methanol or ethanol and the acid is added at a temperature of about 0° C. to about 100° C., preferably at ambient temperature. The resulting salt either precipitates or may be brought out of solution by addition of a less polar solvent. Suitable acids for preparing the acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like as well as inorganic acids, e.g., hydrochloric acid, hydrobromic acid, sulfuric acid, nitric acid, phosphoric acid, and the like. An acid addition salt may be reconverted into the free base by treatment with a suitable base. Basic addition salts of an active agent having an acid moiety (e.g., carboxylic acid group or hydroxyl group) are prepared in a similar manner using a pharmaceutically acceptable base. Suitable bases include both inorganic bases, e.g., sodium hydroxide, potassium hydroxide, ammonium hydroxide, calcium hydroxide, magnesium hydroxide, and the like, as well as organic bases such as trimethylamine, or the like. Preparation of esters involves functionalization of hydroxyl and/or carboxyl groups that may be present within the molecular structure of the drug. The esters are typically acyl-substituted derivatives of free alcohol groups, i.e., moieties which are derived from carboxylic acids of the formula RCOOH where R is alkyl, and preferably is lower, i.e., C₁ to C₆, alkyl. Esters can be reconverted to the free acids, if desired, by using conventional hydrogenolysis or hydrolysis procedures. Preparation of amides and prodrugs can be carried out in an analogous manner. Other derivatives of the active agents may be prepared using standard techniques known to those skilled in the art of synthetic organic chemistry, or may be deduced by reference to the pertinent literature and texts.

The active agent may also be administered in the form of an isolated stereoisomer or enantiomer as individual stereoisomers or enantiomers may have unique or beneficial properties that make that individual isomer particularly suited for administration using the composition and drug delivery systems of the invention.

The amount of the additional active agent represents a therapeutically effective amount, and will of course vary depending on the particular additional agent, but will generally be in the range of about 0.1 wt. % to about 50 wt. %, more preferably about 0.1 wt. % to about 30 wt. %, and most preferably about 0.1 wt. % to about 10 wt. % of the composition.

B. The Carrier:

In addition to the active agent the present compositions also include a pharmaceutically acceptable, nonliposomal carrier containing a monohydric alcohol, a penetration enhancer, and a polymer, as will be described in detail below. By “nonliposomal” is meant that the carrier is substantially free of liposomes. As is well known in the art, a liposome is a structure having a lipid bilayer that forms a microscopic sphere enclosing a liquid, e.g., aqueous, core. The bilayer is typically formed from phospholipids, although other materials may be used. While not wishing to be bound by theory, liposomal and other “barrier-forming” carriers are believed to delay the onset of anesthesia because the active agent has to penetrate through a membrane or wall, e.g., through the lipid bilayer of a liposome. In contrast, nonliposomal carriers have no such barrier preventing the active agent from directly contacting the target site, e.g., skin. It must be noted, however, that liposomal materials such as phospholipids can be present in the compositions so long as the composition is substantially free of liposomesper se. In this context, “substantially free of liposomes” is meant that less than about 40 wt. %, more preferably less than about 10 wt. %, of the active agent is encapsulated within liposomes.

Applicants have additionally found that the incorporation of a monohydric alcohol in a local anesthetic-containing composition provides the composition with superior performance characteristics. While not wishing to be bound by theory, applicants believe that the monohydric alcohol increases the carrier's overall solubilizing capacity while enhancing the penetration of the local anesthetic agent. Moreover, relatively large quantities of a monohydric alcohol result in film-forming compositions, such that an anesthetic-containing film is formed when the monohydric alcohol volatilizes.

Preferred monohydric alcohols include, without limitation, C₁-C₁₈ branched, linear, cyclic, saturated and unsaturated monohydric alcohols. Among unbranched monohydric alcohols, methanol, ethanol, denatured ethanol, propanol, butanol, pentanol, hexanol, heptanol, octanol, nonanol, decanol, undecanol, dodecanol (i.e., lauryl alcohol), tridecanol, tetradecanol (i.e., myristyl alcohol), pentadecanol and hexadecanol (i.e., palmityl alcohol) are preferred. Other preferred monohydric alcohols include isopropyl alcohol, isobutyl alcohol, s-butyl alcohol, t-butyl alcohol, cyclohexanol, phenol, benzyl alcohol, and so forth. The monohydric alcohol can be optionally substituted with 1 to 4 substituents such as halo, lower alkoxy, thiol, and so on. Of course, combinations of any of the foregoing monohydric alcohols or additional alcohols may be included in the compositions and systems described herein.

The amount of monohydric alcohol in the composition is based, at least in part, on the type of formulation desired. Thus, for example, relatively lower amounts, i.e., in the range of about 1 wt. % to about 40 wt. %, are present in gel formulations. For film-forming compositions, the monohydric alcohol plays a role as a film-forming agent in the composition, in which case the composition may additionally contain other film-forming adjuvants such as dimethylsiloxane, dimethylsulfoxide or a combination thereof. For film-forming compositions, the monohydric alcohol is present in the composition in an amount of in the range of about 40 wt. % to about 90 wt. %. Preferably the amount of the film-forming agent is in the range of about 50 wt. % to about 80 wt. % of the composition.

For film-forming compositions, the volatility of the monohydric alcohol is such that upon application to a body surface, at least a fraction of the monohydric alcohol volatilizes. Consequently, when a film-forming composition is desired, the monohydric alcohol is preferably volatile, i.e., having a relatively low vapor pressure. Specifically, the vapor pressure of the monohydric alcohol in a film-forming composition is preferably less than about 75 kPa, more preferably less than about 50 kPa, and most preferably less than about 25 kPa at 25° C. As a lower limit, the vapor pressure of the monohydric alcohol in the film-forming composition will typically be higher than about 0.001 kPa at 25° C. Vapor pressures of monohydric alcohols can be determined experimentally or may be found in the relevant texts, e.g., CRC Handbook of Chemistry and Physics, 81st Ed., Lide, Ed. (Boca Raton: CRC Press, 2000). Although most alcohols will have some degree of volatility, preferred monohydric alcohols in the film-forming compositions include methanol, ethanol, denatured ethanol, propanol alcohol, isopropyl alcohol, butanol, isobutyl alcohol, sec-butyl alcohol, t-butyl alcohol, cyclohexyl alcohol, phenol, benzyl alcohol, pentanol, hexanol, menthol, and combinations thereof. Of these, particularly preferred monohydric alcohols are the lower (C₁-C₄) monohydric alcohols, i.e., methanol, ethanol, denatured ethanol, propanol, isopropyl alcohol, butanol, isobutyl alcohol, s-butyl alcohol, t-butyl alcohol, and combinations thereof.

In order to provide enhanced penetration through the skin, the compositions and systems also include one or more penetration enhancers. Suitable enhancers include, for example, the following: sulfoxides such as dimethylsulfoxide (DMSO) and decylmethylsulfoxide (C₁₀MSO); ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, poloxamer (231, 182, 184), poly(oxyethylene) sorbitans, e.g., Tween® (20, 40, 60, 80) and lecithin (see, e.g., U.S. Pat. No. 4,783,450); pentadecalactone; methyl nicotinate; cholesterol; bile salts; fatty acids such as lauric acid, oleic acid and valeric acid; fatty acid esters such as isopropyl myristate, isopropyl palmitate, methylpropionate and ethyl oleate; polyols and esters thereof such as propylene glycol, propylene glycol monolaurate, ethylene glycol, glycerol, butanediol, polyethylene glycol and polyethylene glycol monolaurate (PEGML; see, e.g., U.S. Pat. No. 4,568,343); phospholipids such as phosphatidyl choline, phosphatidyl ethanolamine, dioleoylphosphatidyl choline (DOPC), dioleoylphosphatidyl glycerol (DOPG) and dioleoylphoshatidyl ethanolamine (DOPE); amides and other nitrogenous compounds such as urea, dimethylacetamide (DMA), dimethylformamide (DMF), 2-pyrrol idone, 1-methyl-2-pyrrolidone, ethanolamine, diethanolamine and triethanolamine; terpenes; alkanones; cyclodextrins and substituted cyclodextrins such as dimethyl-β-cyclodextrin, trimethyl-β-cyclodextrin and hydroxypropyl-β-cyclodextrin; and organic acids, particularly salicylic acid and salicylates, citric acid, and succinic acid. Particularly preferred penetration enhancers herein are hydroxypropyl-β-cyclodextrin, isopropyl myristate, oleic acid, pentadecalactone, propylene glycol, propylene glycol monolaurate and triethanolamine. Combinations of any of the foregoing enhancers are contemplated as well.

The amount of the penetration enhancer in the composition is an effective enhancing amount. Generally an effective amount of an enhancer is in the range of about 0.1 wt. % to about 20 wt. %, more preferably from about 1 wt. % to about 10 wt. %, of the dry composition

The carrier also includes a polymer, and the type of polymer selected influences the characteristics and performance of the composition or drug delivery system. Hydrophobic gels will contain different polymers than hydrophilic gels, and water-soluble film-forming compositions will contain polymers other than those used in water-insoluble film-forming compositions. The polymers used in the present compositions and delivery systems include hydrophilic polymers, hydrophobic polymers and combinations thereof.

The definitions of “hydrophobic” polymers and “hydrophilic” polymers are based on the amount of water vapor absorbed by polymers at 100% relative humidity (“rh”). According to this classification, hydrophobic polymers absorb only up to 1 wt. % water at 100% rh, while moderately hydrophilic polymers absorb 1-10 wt. % water, hydrophilic polymers are capable of absorbing more than 10 wt. % of water, and hygroscopic polymers absorb more than 20 wt. % of water.

Gels are semisolid, suspension-type systems. Single-phase gels contain polymers distributed substantially uniformly throughout a carrier. In the present gel compositions, as discussed above, the amount of the monohydric alcohol is generally in the range of about 1 wt. % to about 40 wt. %. The type of gel, e.g., hydrophobic or hydrophilic, will largely depend upon the type of polymer used. In order to prepare a substantially uniform gel, the components of the carrier are thoroughly mixed, followed by addition of the active agent, which is then blended with the carrier.

Examples of suitable hydrophilic polymers used in hydrophilic gels of the invention include, but are not limited to: poly(N-vinyl lactams) such as polyvinyl pyrrolidone, poly(N-vinyl-2-valerolactam), and N-vinyl-2-caprolactam (optionally copolymerized with one or more second monomers such as N,N-dimethylacrylamide, acrylic acid, methacrylic acid, hydroxyethylmethacrylate, acrylamide, 2-acrylamido-2-methyl-1-propane sulfonic acid, and vinyl acetate); polyethylene glycol; poly(ethylene oxide-co-propylene oxide); polyvinyl alcohol; polyvinyl acetate; polylactide; poly(lactide-co-glycolide); polysorbate; poly(oxyethylated) glycerol; poly(oxyethylated) sorbitol; poly(oxyethylated) glucose; cellulosic polymers such as hydroxyethyl cellulose, hydroxypropyl cellulose, and hydroxypropyl methylcellulose; carbomers, i.e., hydroxylated vinylic polymers also referred to as “interpolymers,” which are prepared by crosslinking a monoolefinic acrylic acid monomer with a polyalkyl ether of sucrose (commercially available under the trademark Carbopol® from the B.F. Goodrich Chemical Company); acrylamide-sodium acrylate copolymers; gelatin; vegetable polysaccharides, such as alginates, pectins, carrageenans, or xanthan gum; starch and starch derivatives; galactomannan and galactomannan derivatives; and acrylate polymers, generally formed from acrylic acid, methacrylic acid, methyl acrylate, ethyl acrylate, methyl methacrylate, ethyl methacrylate, and/or other vinyl monomers. Suitable acrylate polymers are those copolymers available under the tradename “Eudragit” from Rohm Pharma (Germany). Preferred acrylate polymers are copolymers of methacrylic acid and methyl methacrylate, such as the Eudragit L and Eudragit S series polymers.

Preferred polymers for providing hydrophilic gels are the following: poly(N-vinyl lactams), particularly polyvinyl pyrrolidone; polyvinyl alcohol; polyvinyl acetate; cellulosic polymers; acrylate polymers; carbomers; gelatin; alginates; pectins; carageenan; tragacanth; xanthan gum; starches; and galactomannans, with carageenans particularly preferred. As used herein, the term “carrageenan” means a mixture of sulfated polysaccharides extracted from red seaweed (Rhodophyceae) having the ability to form gels. Carrageenan has been used extensively in the food industry and is available from commercial suppliers such as FMC Corp. (Philadelphia, Pa.).

The composition forms a hydrophobic or oily gel when the polymer in the composition is hydrophobic in nature. Hydrophobic polymers include, by way of example, butyl rubber, which, as well known in the art, is an isoprene-isobutylene copolymer typically having an isoprene content in the range of about 0.5 to 3 wt. %, or a vulcanized or modified version thereof, e.g., a halogenated (brominated or chlorinated) butyl rubber. A preferred polymer is butyl rubber crosslinked with polyisobutylene. Other suitable hydrophobic polymers include, by way of example and not limitation, ethylene-propylene-styrene terpolymers, butylene-ethylene-styrene terpolymers, natural rubber adhesives, vinyl ether polymers, polysiloxanes, polyisoprene, butadiene acrylonitrile rubber, polychloroprene, atactic polypropylene, ethylene-propylene-diene terpolymers (also known as “EPDM” or “EPDM rubber”) (available as Trilene® 65 and Trilene® 67 from Uniroyal Chemical Co., Middlebury, Conn.), and butylene-ethylene-diene terpolymers. Particularly preferred hydrophobic polymers are ethylene-propylene-styrene terpolymers, butylene-ethylene-styrene terpolymers, and butyl rubber.

Additionally, the hydrophobic gel compositions preferably include an oil, a fatty acid ester, or both. The addition of an oil and/or fatty acid ester enhances the hydrophobicity of the gel without affecting the stability of the gel. As the amount of the oil and/or fatty acid ester in the composition increases, so does the hydrophobicity of the composition.

The oil may be naturally occurring, of vegetable, mineral or animal origin, or synthetic, and may be a single oil or comprised of a mixture of oils. Vegetable oils are derived from various plants and are generally produced by extraction or pressing processes, and include castor oil, linseed oil, sunflower oil, soybean oil, olive oil, peanut oil, rapeseed oil, corn oil, safflower oil, cottonseed oil, coconut oil, palm oil, palm kernel oil, etc. Mineral oils are derived from petroleum and are recovered through various refining processes, and include white mineral oil, paraffin oil, petrolatum and the like. Animal oils are derived from the organs and tissues of animals and may be collected through extraction, heating and/or expressing processes, and include lanolin, fatty acid esters, fish oil, whale oil, fish liver oil, seal oil, squalene, and so forth. Synthetic oils include silicone oils, e.g., dimethylpolysiloxane, cyclic silicones, methylphenyl-polysiloxane, etc. It is particularly preferred that the oil, when present, is a mineral oil. Any combination of oils may be used as well.

The fatty acid ester is typically a lower alkyl ester of a C₈ to C₁₈ fatty acid. Although any fatty acid ester may be used, preferred fatty acid esters are ethyl caprate, ethyl caprylate, ethyl oleate, ethyl laurate, ethyl linoleate, ethyl myristate, ethyl palmitate, ethyl stearate, isopropyl caprate, isopropyl caprylate, isopropyl oleate, isopropyl laurate, isopropyl linoleate, isopropyl myristate, isopropyl palmitate, butyl caprate, butyl caprylate, butyl oleate, butyl laurate, butyl linoleate, butyl myristate, butyl palmitate, and butyl stearate. A particularly preferred fatty acid ester is isopropyl palmitate.

As described herein, some of the present compositions, when placed on a body surface, will form a film containing the local anesthetic agent. Less bulky and more discreet than patches, the films obtained from these compositions still provide effective topical administration of a local anesthetic agent. Furthermore, the monohydric alcohol and any other solvents that may be present (e.g., water, acetone, and so forth) appear to initially enhance penetration, providing a rapid onset of anesthesia before evaporating completely. The film-forming composition can advantageously be sprayed onto skin using conventional means, e.g., an atomizer, spray bottle, or pressurized can, thereby avoiding direct contact with the body surface. The film-forming composition can be applied manually as well. Generally, although not necessarily, the film forms within about fifteen minutes, more preferably within about five minutes and most preferably within about one minute of application to the body surface. Moreover, the film that is formed has a thickness of from about 0.01 mm to about 2 mm, with a thickness of from about 0.1 to about I mm being preferred.

The polymer used in water-soluble, film-forming compositions may be any polymer that will form a water-soluble film following application of the composition to a body surface. A water-soluble film can be removed easily with the application of water and gentle agitation. Among other advantages, water-soluble films are easily removable, thereby providing a facile method for removing the film and subsequently discontinuing treatment.

Preferred polymers for water-soluble, film-forming compositions generally include the hydrophilic polymers set forth above, providing that the resulting composition forms a water-soluble film upon evaporation of water and any other solvents that may be present. Such polymers include, for example, certain cellulosic polymers, e.g., hydroxypropyl cellulose; acrylate polymers; carbomers; gelatin; alginates; pectins; carrageenan; xanthan gum; starches; galactomannans; and poly(N-vinyl lactams), e.g., polyvinyl pyrrolidone and poly(N-vinyl caprolactam). Particularly preferred polymers for use in the water-soluble, film-forming compositions are poly(N-vinyl lactams). In addition, combinations of any of the foregoing may be used.

Water-insoluble film-forming compositions include a polymer that will form a water-insoluble film upon application of the composition to a body surface. One advantage of water-insoluble films is that they resist removal, even with application of copious amounts of water and agitation. Accordingly, these films remain in place even in the presence of aqueous liquids such as sweat. Preferred polymers for use in compositions that form water-insoluble films generally include the hydrophobic polymers discussed above so long as the resulting composition forms a water-insoluble film upon evaporation of water and any other solvents that may be present. Preferred polymers, in this embodiment, are cellulose esters, e.g., cellulose acetate butyrate, cellulose acetate, cellulose acetate phthalate, and cellulose acetate propionate, and cellulose ethers, e.g., ethyl cellulose and methyl cellulose. Combinations of the polymers may also be used.

Water-resistant films may also be obtained from the present compositions. “Water-resistant” films are those films that are only partially resistant to removal with water and gentle agitation. The polymer in this embodiment is preferably a polymer of an amino acid. Thus, preferred polymers are proteins, with zein, a corn protein, being particularly preferred.

Other suitable hydrophobic and hydrophilic polymers can also be used in the film-forming compositions, and the invention is not limited in this regard. Any particular polymer can be tested for suitability in a film-forming composition of the invention by preparing a composition using the polymer of interest along with the other components, e.g., the monohydric alcohol, anesthetic agent, penetration enhancer, and so forth, and determining whether a film forms following application to a body surface. Moreover, the type of film formed, e.g., a water-soluble film, a water-insoluble film or water-resistant film, can be readily determined by detecting substantially all, some, or substantially none of the film following gentle agitation and washing with water.

All of the components of the compositions described herein (including the compositions used in the systems) are commercially available or may be readily synthesized from commercially available materials. For example, in the hydrophobic gel, a mixture of mineral oil, ethylene-propylene-styrene copolymer, and butylene-ethylene-styrene copolymer is available under the tradename Versagel® M (Penreco, Karns City, Pa.). In addition, a mixture of isopropyl palmitate, ethylene-propylene-styrene copolymer, and butylene-ethylene-styrene copolymer is available under the tradename Versagel® MP (Penreco, Karns City, Pa.).

The compositions described herein (including the compositions incorporated in drug delivery systems) may also contain one or more pharmaceutically acceptable excipients. Preferred excipients include plasticizers, antioxidants, stabilizers, surfactants, solvents, preservatives, pH regulators, softeners, thickeners, colorants or a combination thereof. Any additives should not significantly interfere with the desired chemical and physical properties of the composition or delivery system in which they are contained.

Incorporation of an antioxidant is optional but preferred. The antioxidant serves to enhance the oxidative stability of the composition. Heat, light, impurities, and other factors can all result in oxidation of the components of the composition. Thus, ideally, antioxidants should protect against light-induced oxidation, chemically induced oxidation, and thermally induced oxidative degradation during processing and/or storage. Oxidative degradation, as will be appreciated by those in the art, involves generation of peroxy radicals, which in turn react with organic materials to form hydroperoxides. Primary antioxidants are peroxy free radical scavengers, while secondary antioxidants induce decomposition of hydroperoxides, and thus protect a material from degradation by hydroperoxides. Most primary antioxidants are sterically hindered phenols, and preferred such compounds for use herein are tetrakis [methylene (3,5-di-tert-butyl-4-hydroxyhydrocinnamate)] methane (e.g., Irganox® 1010, from Ciba-Geigy Corp., Hawthorne, N.Y.) and 1,3,5-trimethyl-2,4,6-tris [3,5-di-t-butyl-4-hydroxy-benzyl] benzene (e.g., Ethanox® 330, from Ethyl Corp.). A particularly preferred secondary antioxidant that may replace or supplement a primary antioxidant is tris(2,4-di-tert-butylphenyl)phosphite (e.g., Irgafos® 168, Ciba-Geigy Corp.). Other antioxidants, including but not limited to multi-functional antioxidants, are also useful herein. Multifunctional antioxidants serve as both a primary and a secondary antioxidant. Irganox® 1520 D, manufactured by Ciba-Geigy is one example of a multifunctional antioxidant. Vitamin E antioxidants, such as that sold by Ciba-Geigy as Irganox® E17, are also useful in the present composition. Other suitable antioxidants include, without limitation, ascorbic acid, ascorbic palmitate, tocopherol acetate, propyl gallate, butylhydroxyanisole (BHA), butylated hydroxytoluene (BHT), bis(1,2,2,6,6-pentamethyl-4-piperidinyl)-(3,5-di-tert-butyl-4-hydroxybenzyl)butylpropanedioate, (available as Tinuvin®144 from Ciba-Geigy Corp.) or a combination of octadecyl 3,5-di-tert-butyl-4-hydroxyhydro-cinnamate (also known as octadecyl 3-(3′,5′-di-tert-butyl-4′-hydroxyphenyl)propionate) (available as Naugard® 76 from Uniroyal Chemical Co., Middlebury, Conn.) and bis(1,2,2,6,6-pentamethyl-4-piperidinylsebacate) (available as Tinuvin®765 from Ciba-Geigy Corp.). Preferably, the antioxidant is present in amount up to about 2 wt. % of the composition; typically, the amount of antioxidant is in the range of about 0.05 wt. % to 1.5 wt. %.

Preservatives serve to at least partially inhibit the growth of microbes. Preservatives include, by way of example, p-chloro-m-cresol, phenylethyl alcohol, phenoxyethyl alcohol, chlorobutanol, 4-hydroxybenzoic acid methylester, 4-hydroxybenzoic acid propylester, benzalkonium chloride, cetylpyridinium chloride, chlorohexidine diacetate or gluconate, ethanol, and propylene glycol.

Compounds useful as pH regulators include, but are not limited to, glycerol buffers, citrate buffers, borate buffers, phosphate buffers, or citric acid-phosphate buffers may also be included so as to ensure that the pH of the hydrogel composition is compatible with that of an individual's body surface. Solutions of acids and bases, e.g., a solution of sodium hydroxide, can be used to bring pH of the desired composition to a suitable range. In particular, the solution of sodium hydroxide (0.075M) can be used to bring the pH of present compositions to about 9.0. It is believed that compositions having a pH about 9.0 or higher do not require additional antimicrobial preservatives.

Suitable softeners include, by way of example, the following: citric acid esters, such as triethylcitrate or acetyl triethylcitrate; tartaric acid esters such as dibutyltartrate; glycerol esters such as glycerol diacetate and glycerol triacetate; phthalic acid esters such as dibutyl phthalate and diethyl phthalate; and/or hydrophilic surfactants, preferably hydrophilic non-ionic surfactants, such as, for example, partial fatty acid esters of sugars, polyethylene glycol fatty acid esters, polyethylene glycol fatty alcohol ethers, and polyethylene glycol sorbitan-fatty acid esters.

III. Drug Delivery Systems

The invention also provides a drug delivery system for topical administration of a local anesthetic agent. In one embodiment, the system is a device in the form of a laminated composite comprising: (a) a drug reservoir layer containing a pharmaceutical composition of (i) a therapeutically effective amount of a local anesthetic agent, (ii) a monohydric alcohol, and (iii) an effective enhancing amount of a penetration enhancer; and (b) a backing layer laminated to the drug reservoir layer that serves as the outer surface of the device following application to a patient's body surface.

In the manufacture of such systems, the reservoir layer may be cast or extruded onto a backing layer or release liner of such a system and serves as the skin contacting face of the “patch.” Alternatively, the drug reservoir layer may be contained within the system, with a conventional bioadhesive laminated thereto.

The drug reservoir layer contains a quantity of a local anesthetic agent effective to provide the desired dosage over a predetermined delivery period. The drug reservoir layer optionally contains excipients such as colorants, thickening agents, stabilizers, surfactants and the like. Reference is made to the discussion of local anesthetic agents, penetration enhancers, and optional excipients discussed above.

The drug reservoir layer can be a polymeric matrix of a pharmaceutically acceptable bioadhesive material that provides the means to affix the system to a body surface. In such a case, the device is “monolithic,” meaning that a single layer serves as both the drug reservoir and the bioadhesive. Alternatively, the bioadhesive material may be an additional layer defining the basal surface of the device. The drug reservoir may also be comprised of a hydrogel. The drug reservoir can also be a sealed compartment containing the pharmaceutical local anesthetic agent and other components in a liquid or gel formulation. Optionally, more than one drug reservoir layer can be present, each layer containing the same active agent or a different active agent.

The backing layer functions as the primary structural element of the system and preferably provides flexibility as well as protection of the underlying system. The material used for the backing layer should be inert and incapable of absorbing drug, enhancer or other components of the composition. Also, the material used for the backing layer should permit the system to follow the contours of the body surface and be worn comfortably. For example, the material should permit the device to be used on areas of skin such as atjoints or other points of flexure that are normally subjected to mechanical strain with little or no likelihood of the device disengaging from the skin due to differences in the flexibility or resiliency of the skin and the device. Examples of materials useful for the backing layer are polyesters, polyethylene, polypropylene, polyurethanes and polyether amides. The layer is preferably in the range of about 15 microns to about 250 microns in thickness, and may, if desired, be pigmented, metallized, or provided with a matte finish suitable for writing. The layer is preferably although not necessarily nonocclusive (or “breathable”), i.e., is preferably permeable to moisture.

Additional layers, e.g., intermediate fabric layers and/or rate-controlling membranes, may also be present. Fabric layers may be used to facilitate fabrication, while a rate-controlling membrane may be used to control the rate at which a component permeates out of the device. The component may be a drug, a penetration enhancer, or some other component contained in the drug delivery system.

In these systems, it may be desirable to include a rate-controlling membrane in the system on the body surface side of the drug reservoir. The materials used to form such a membrane are selected to limit the flux of one or more components contained in the drug formulation, and the membrane may be either microporous or dense. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, polysiloxane-polycarbonate block copolymer and the like.

For adhesive purposes, an acceptable bioadhesive material may be present in the drug reservoir or in a separate layer. Preferred adhesive materials include crosslinked polyisobutylene, butyl rubber, natural rubber adhesives, vinyl ether polymers, polysiloxanes, polyisoprene, butadiene acrylonitrile rubber, polychloroprene, atactic polypropylene, ethylene-propylene-diene terpolymers, and combinations thereof. Other suitable adhesives will be known to those of ordinary skill in the art and/or are described in the pertinent texts and literature. See, for example, the Handbook of Pressure-Sensitive Adhesive Technology, 2nd Ed., Satas, Ed. (New York: Von Nostrand Reinhold, 1989).

The invention also provides a drug delivery system for administration of a local anesthetic agent to a mucosal surface, wherein the system comprises: (a) a water-soluble drug reservoir layer containing a pharmaceutical composition of (i) a therapeutically effective amount of a local anesthetic agent, (ii) a monohydric alcohol, (iii) an effective enhancing amount of a penetration enhancer and (iv) a polymer; and (b) an optional hydrophobic layer that serves as the outer surface of the device following application to the mucosal surface.

The various components of the system are as described above, providing that the drug reservoir is water soluble and that the system is preferably composed of a single layer (not including a release liner).

The drug delivery system for mucosal administration will preferably be used for buccal delivery, but the system may be applied to any mucosal surface for which local anesthesia is desired. Particularly for buccal delivery, the local anesthetic agent may be released from the system into the general region of the oral cavity, thereby reaching not only the area where the system is applied, but also areas proximal to the site of application. Although any polymer may be used that is suited for transmucosal delivery, preferred polymers are poly(N-vinyl lactams), particularly polyvinyl pyrrolidone and poly(N-vinyl caprolactam), polyethylene glycol, and combinations thereof.

Optionally, the transmucosal system includes a hydrophobic, water-resistant layer, which may or may not contain an active agent. The hydrophobic layer preferably, although not necessarily, comprises a cellulose ester such as cellulose acetate butyrate, cellulose acetate, cellulose acetate phthalate, cellulose acetate propionate, and combinations thereof.

IV. Utility and Administration:

The invention also provides a method for administering a local anesthetic agent to a patient to treat or prevent pain. The method involves topically administering a pharmaceutical composition as described herein. The present method may be used to treat patients suffering from oral pain, including, but not limited to, a cold sore, canker sore, gum sore, gum injury, tooth ache, cough, sore throat or a combination thereof. Additionally, the method may be used to treat patients suffering from pain associated with a skin condition or disorder, e.g., an insect bite, muscle pain, arthritis, allergic reaction, rash (e.g., a rash caused by poison oak or poison ivy), itch, blister, sore nail, corn, mechanical puncture (e.g., catheterization and needle injection), laser treatment, or any combination thereof.

The method may also be used to treat patients suffering from breakthrough pain, migraine, neuropathic pain, and anginal pain. In addition, the compositions and systems of the invention may be administered with a wound dressing to treat burns, wounds and scrapes.

Advantageously, the compositions and drug delivery systems described herein can also be used as part of a pre-treatment regimen used to prevent or minimize the pain associated with other topical therapies, medical procedures or cosmetic procedures.

The compositions and drug delivery systems described herein have many advantages. Anesthesia is provided quickly, within about 15 to 30 minutes following application. Furthermore, penetration of the local anesthetic agent is enhanced, e.g., the local anesthetic agent can penetrate at least about 5 mm into the body surface. In addition, depending upon the anesthetic agent used, effective anesthesia can be maintained for at least 4 hours, and more preferably at least 6 hours.

The amount of the active agent administered depends upon the age, weight, and general condition of the subject, the severity of the condition being treated, and the judgment of the prescribing physician or attending clinician. Therapeutically effective amounts will be known to those skilled in the art and/or are described in the pertinent reference texts and literature. An effective amount of the composition may be administered by placing an appropriate amount, e.g., about 0.1 g to about 5 g of the composition, to the affected area. Alternatively, when a drug delivery system is used, the system will contain the effective amount and may applied to the affected area.

Generally, the effective amount will be in the range of about 0.01 mg to about 100 mg, more preferably about 0.1 mg to about 25 mg, and most preferably about 0.1 mg to about 10 mg of the active agent. Administration of the active agent can be carried out once, twice, three times or four times daily. Alternatively, or in addition to regularly scheduled doses, administration may be carried out on an “as needed” basis, or using a drug delivery system adapted to provide sustained drug delivery over an extended time period. The total daily dose, however, should generally not exceed about 5,000 mg of the active agent.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications and publications mentioned herein are hereby incorporated by reference in their entireties.

V. Experimental

The practice of the invention will employ, unless otherwise indicated, conventional techniques of pharmaceutical formulating and the like, which are within the skill of the art. Such techniques are fully explained in the literature. See for, example, Remington: The Science and Practice of Pharmacy, Twentieth Edition (Easton, Pa.: Mack Publishing Co., 2000).

In the following examples, efforts have been made to ensure accuracy with respect to numbers used (e.g., amounts, temperatures, etc.) but some experimental error and deviation should be accounted for. Unless otherwise indicated, temperature is in degrees C and pressure is at or near atmospheric pressure at sea level. All reagents and formulation components were obtained commercially unless otherwise indicated.

### EXAMPLE 1

A pin-prick model was used to evaluate the efficacy of a carrageenan-based gel according to the present invention against the ELA-MAX® 5 brand of topical anesthetic cream, a cream commercially available from Ferndale Laboratories, Ferndale Mich. For each of the tested 10, 30, 60 and 180 time intervals, 0.025 g of formulation was applied to a 2 cm² area on the ventral forearm of healthy volunteers. The formulation was allowed to remain in place until administration of the pin pricks at the tested time interval (i.e., 10, 20, 60 or 180 minutes), and was then removed from the forearm immediately prior to the administration of the pin pricks. A fifth series of pin pricks was administered at 60 minutes following the 180 minute interval, thereby providing data at 240 minutes. The pin used to administer the pricks has a diameter of 0.2 mm and a length of 1 mm. As a control, an untreated site on the ventral forearm was also pricked with the pin.

The ELA-MAX® 5 brand of topical anesthetic cream contains lidocaine (5%) and uses a liposomal delivery system. The components of the carrageenan-based gel used in the example are provided in Table 1.

TABLE 1
Components of the Carrageenan-Based Gel

| Component | Amount (wt. %) |
|---|---|
| Carrageenan | 3% |
| Tetracaine | 2% |
| Hydroxypropyl-β-cyclodextrin | 2% |
| Lauryl alcohol | 2% |
| Propylene glycol monolaurate | 2% |
| pH 9 solution (NaOH 0.075M) | q.s. to 100% |

The carrageenan-based gel was prepared using conventional techniques. Briefly, the carrageenan, tetracaine, hydroxypropyl-β-cyclodextrin, lauryl alcohol, and propylene glycol monolaurate were measured, combined, and thoroughly mixed. Thereafter, a pH 9 solution (NaOH 0.075M) was added in amount sufficient to bring the total to 100 %.

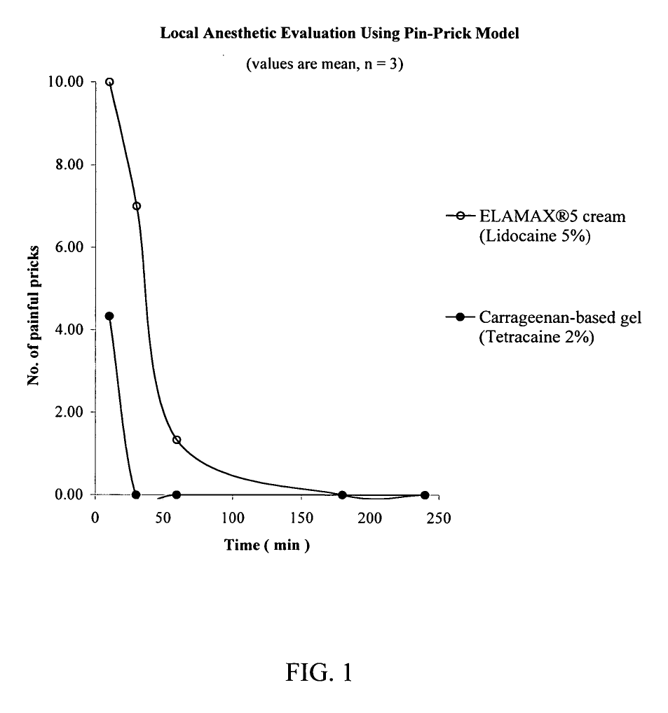

At untreated sites, 100% of the pin pricks were felt as painful. As demonstrated in FIG. 1, the anesthesia achieved by the carrageenan-based gel (a hydrophilic carrier) was dramatically higher than that of the commercially available ELA-MAX®5 brand of topical anesthetic cream.

### EXAMPLE 2

The procedure of Example 1 was repeated except that EMLA® brand of topical cream was used in place of ELA-MAX® brand of topical anesthetic cream. The EMLA® brand of topical cream was obtained from AstraZeneca, Wilmington Del. As described in the packaging provided by the manufacturer, each gram of EMLA® brand of topical cream contains lidocaine (25 mg), prilocaine (25 mg), polyoxyethylene fatty acid esters, carboxypolymethylene, sodium hydroxide and purified water.

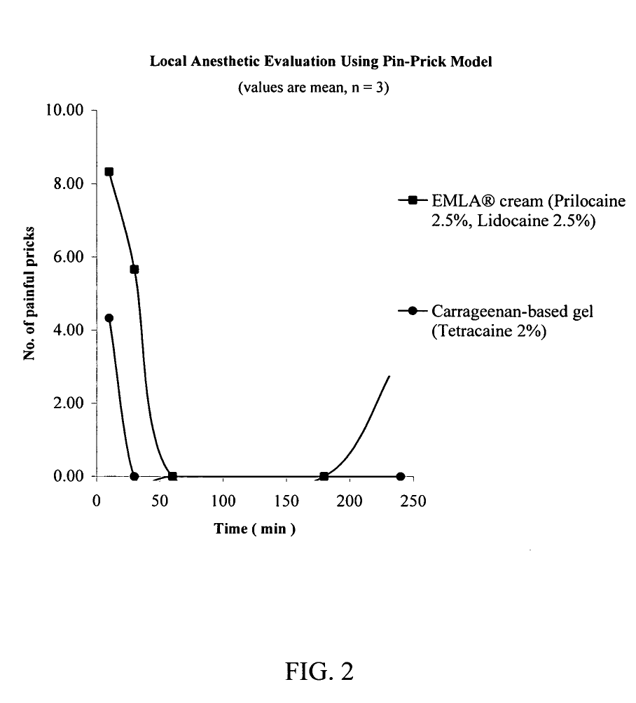

At untreated sites, 100% of the pin pricks were felt as painful. As demonstrated in FIG. 2, the anesthesia achieved by the carrageenan-based gel (a hydrophilic carrier) was dramatically higher than that of the commercially available EMLA® 5 brand of topical cream.

### EXAMPLE 3

The procedure of Example 1 was repeated except that AMETOP® brand of topical anesthetic cream was used in place of ELA-MAX®5 brand of topical anesthetic cream. The AMETOP® brand of topical anesthetic cream was obtained from Smith and Nephew, London, United Kingdom.

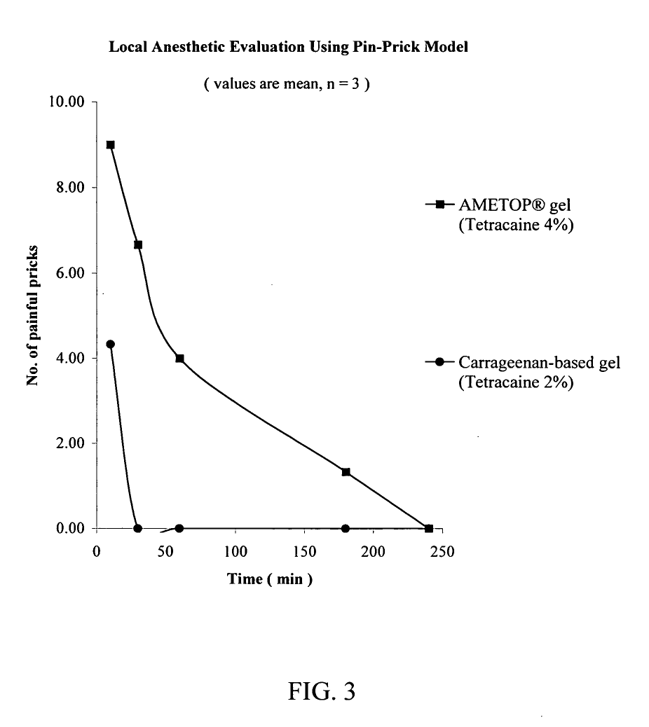

At untreated sites, 100% of the pin pricks were felt as painful. As demonstrated in FIG. 3, the anesthesia achieved by the carrageenan-based gel (a hydrophilic carrier) was dramatically higher than that of the commercially available AMETOP® brand of topical anesthetic cream.

### EXAMPLE 4

Propylene glycol (3 gm) and 10 grams of polyvinyl pyrrolidone are dissolved and mixed with 70 ml of ethanol. Ten grams of lidocaine are then added and mixed to form a film-forming composition. Upon application to a body surface, the composition forms a film and alleviates the pain associated with a wound. When washed with water, the film is easily removed.

1. A pharmaceutical composition comprising (a) a therapeutically effective amount of a local anesthetic agent and (b) a pharmaceutically acceptable, nonliposomal carrier comprised of a monohydric alcohol, an effective penetration enhancing amount of a penetration enhancer, and a polymer selected from the group consisting of hydrophilic polymers, hydrophobic polymers and combinations thereof, wherein local anesthetic activity is provided within about thirty minutes of application of the composition to a patient's body surface.

2. The composition of claim 1, wherein the local anesthetic agent is selected from the group consisting of acetamidoeugenol, alfadolone acetate, alfaxalone, amucaine, amolanone, amylocaine, benoxinate, benzocaine, betoxycaine, biphenamine, bupivacaine, burethamine, butacaine, butaben, butanilicaine, buthalital, butoxycaine, carticaine, 2-chloroprocaine, cocaethylene, cocaine, cyclomethycaine, dibucaine, dimethisoquin, dimethocaine, diperadon, dyclonine, ecgonidine, ecgonine, ethyl aminobenzoate, ethyl chloride, etidocaine, etoxadrol, β-eucaine, euprocin, fenalcomine, fomocaine, hexobarbital, hexylcaine, hydroxydione, hydroxyprocaine, hydroxytetracaine, isobutylp-aminobenzoate, kentamine, leucinocaine mesylate, levoxadrol, lidocaine, mepivacaine, meprylcaine, metabutoxycaine, methohexital, methyl chloride, midazolam, myrtecaine, naepaine, octacaine, orthocaine, oxethazaine, parethoxycaine, phenacaine, phencyclidine, phenol, piperocaine, piridocaine, polidocanol, pramoxine, prilocaine, procaine, propanidid, propanocaine, proparacaine, propipocaine, propofol, propoxycaine, pseudococaine, pyrrocaine, risocaine, salicyl alcohol, tetracaine, thialbarbital, thimylal, thiobutabarbital, thiopental, tolycaine, trimecaine, zolamine, and combinations thereof.

3. The composition of claim 2, wherein the local anesthetic agent is selected from the group consisting of tetracaine, lidocaine, prilocaine, benzocaine, and combinations thereof.

4. The composition of claim 3, wherein the local anesthetic agent is tetracaine.

5. The composition of claim 1, wherein the amount of the local anesthetic agent represents in the range of about 0.1 wt. % to about 50 wt. % of the composition.

6. The composition of claim 5, wherein the amount of the local anesthetic agent represents in the range of about 0.1 wt. % to about 30 wt. % of the composition.

7. The composition of claim 6, wherein the amount of the local anesthetic agent represents in the range of about 0.1 wt. % to about 10 wt. % of the composition.

8. The composition of claim 1, wherein the monohydric alcohol is selected from the group consisting of methanol, ethanol, denatured ethanol, propanol, butanol, pentanol, hexanol, heptanol, octanol, nonanol, decanol, undecanol, lauryl alcohol, tridecanol, myristyl alcohol, pentadecanol and palmityl alcohol, isopropyl alcohol, isobutyl alcohol, sec-butyl alcohol, t-butyl alcohol, cyclohexyl alcohol, phenol, benzyl alcohol, and combinations thereof.

9. The composition of claim 1, wherein the amount of the monohydric alcohol represents in the range of about 1 wt. % to about 40 wt. % of the composition.

10. The composition of claim 1, wherein the penetration enhancer is selected from the group consisting of dimethylsulfoxide, decylmethylsulfoxide, diethylene glycol monoethyl ether, diethylene glycol monomethyl ether, sodium laurate, sodium lauryl sulfate, cetyltrimethyl-ammonium bromide, benzalkonium chloride, poloxamers, poly(oxyethylene) sorbitans, lecithin, pentadecalactone, methyl nicotinate, cholesterol, bile salts, lauric acid, oleic acid, valeric acid, isopropyl myristate, isopropyl palmitate, methylpropionate, ethyl oleate, propylene glycol, propylene glycol monolaurate, ethylene glycol, glycerol, butanediol, polyethylene glycol, polyethylene glycol monolaurate, phosphatidyl choline, phosphatidyl ethanolamine, dioleoylphosphatidyl choline, dioleoylphosphatidyl glycerol, dioleoylphoshatidyl ethanolamine, urea, dimethylacetamide, dimethylformamide, 2-pyrrolidone, 1-methyl-2-pyrrolidone, ethanolamine, diethanolamine, triethanolamine, terpenes, alkanones, cyclodextrins, salicylic acid, citric acid, succinic acid, and combinations thereof.

11. The composition of claim 10, wherein the penetration enhancer is a cyclodextrin.

12. The composition of claim 11, wherein the cyclodextrin is selected from the group consisting of dimethyl-β-cyclodextrin, trimethyl-β-cyclodextrin, hydroxypropyl-β-cyclodextrin, and combinations thereof.

13. The composition of claim 10, wherein the penetration enhancer is selected from the group consisting of cyclodextrins, isopropyl myristate, oleic acid, pentadecalactone, propylene glycol, propylene glycol monolaurate, triethanolamine, and combinations thereof.

14. The composition of claim 1, wherein the composition provides local anesthetic activity within about ten minutes of application of the composition to a patient's body surface

15. The composition of claim 1, wherein the polymer is hydrophilic and the composition is a hydrophilic gel.

16. The composition of claim 15, wherein the polymer is selected from the group consisting of poly(N-vinyl lactams), polyethylene glycol, poly(ethylene oxide-co-propylene oxide), polyvinyl alcohol, polyvinyl acetate, polylactide, poly(lactide-co-glycolide), polysorbate, poly(oxyethylated) glycerol, poly(oxyethylated) sorbitol, poly(oxyethylated) glucose, cellulosic polymers, carbomers, acrylamide-sodium acrylate copolymers, gelatin, alginates, pectins, carrageenans, xanthan gum, starches, galactomannans, acrylate polymers, and combinations thereof.

17. The composition of claim 16, wherein the polymer is selected from the group consisting of polyvinyl pyrrolidone, polyvinyl alcohol, polyvinyl acetate, cellulosic polymers, acrylate polymers, carbomers, gelatin, alginates, pectins, carageenan, tragacanth, xanthan gum, starches, galactomannans, and combinations thereof.

18. The composition of claim 17, wherein the polymer is carrageenan.

19. The composition of claim 18, wherein the monohydric alcohol is lauryl alcohol, the penetration enhancer is comprised of a mixture of propylene glycol monolaurate and hydroxypropyl-β-cyclodextrin, and the local anesthetic agent is tetracaine.

20. The composition of claim 1, wherein the polymer is hydrophobic and the composition is a hydrophobic gel.

21. The composition of claim 20, wherein the polymer is selected from ethylene-propylene-styrene terpolymers, butylene-ethylene-styrene terpolymers, butyl rubber, natural rubber adhesives, vinyl ether polymers, polysiloxanes, polyisoprene, butadiene acrylonitrile rubber, polychloroprene, atactic polypropylene, and combinations thereof.

22. The composition of claim 20, wherein the polymer is selected from the group consisting of ethylene-propylene-styrene terpolymers, butylene-ethylene-styrene terpolymers, butyl rubber, and combinations thereof.

23. The composition of claim 22, further comprising an oil, a fatty acid ester or combination thereof.

24. The composition of claim 1, further comprising a pharmaceutically acceptable excipient.

25. The composition of claim 24, wherein the excipient is selected from the group consisting of antioxidants, stabilizers, surfactants, solvents, preservatives, pH regulators, softeners, colorants and combinations thereof.

26. The composition of claim 1, further comprising an additional active agent.

27. The composition of claim 26, wherein the additional active agent is selected from the group consisting of bacteriostatic and bactericidal compounds, antibiotic agents, topical vasodilators, tissue-healing enhancing agents, amino acids, proteins, proteolytic enzymes, cytokines, polypeptide growth factors and combinations thereof.

28. The pharmaceutical composition of claim 1, wherein the monohydric alcohol is selected to volatilize following application of the composition to a localized region of a patient's body surface, thereby forming a film within the localized region.

29. The composition of claim 28, wherein the monohydric alcohol is selected from the group consisting of methanol, ethanol, denatured ethanol, propanol, isopropyl alcohol, butanol, isobutyl alcohol, s-butyl alcohol, t-butyl alcohol, cyclohexanol, phenol, benzyl alcohol, pentanol, hexanol, menthol, and combinations thereof.

30. The composition of claim 35, wherein the volatile monohydric alcohol is selected from the group consisting of methanol, ethanol, denatured ethanol, propanol, isopropyl alcohol, butanol, isobutyl alcohol, s-butyl alcohol, t-butyl alcohol, and combinations thereof.

31. The composition of claim 28, wherein the monohydric alcohol represents about 40 wt. % to about 90 wt. % of the composition.

32. The composition of claim 28, wherein the film is water soluble.

33. The composition of claim 32, wherein the polymer is selected from the group consisting of hydroxypropyl cellulose, acrylate polymers, carbomers, gelatin, alginates, pectins, carrageenan, xanthan gum, starches, galactomannans, poly(N-vinyl lactams), and combinations thereof.

34. The composition of claim 33, wherein the polymer is a poly(N-vinyl lactam).

35. The composition of claim 34, wherein the poly(N-vinyl lactam) is selected from the group consisting of polyvinyl pyrrolidone, poly(N-vinyl caprolactam), and combinations thereof.

36. The composition of claim 28, wherein film is water insoluble.

37. The composition of claim 36, wherein the polymer is a cellulose ester.

38. The composition of claim 37, wherein the cellulose ester is selected from the group consisting of cellulose acetate butyrate, cellulose acetate, cellulose acetate phthalate, cellulose acetate propionate, and combinations thereof.

39. The composition of claim 36, wherein the polymer is a cellulose ether.

40. The composition of claim 39, wherein the cellulose ether is selected from the group consisting of ethyl cellulose, methyl cellulose, and combinations thereof.

41. The composition of claim 28, wherein the film is water resistant.

42. The composition of claim 41, wherein the polymer is a protein.

43. The composition of claim 42, wherein the protein is zein.

44. The composition of claim 28, further comprising a film-forming adjuvant.

45. The composition of claim 44, wherein the film-forming adjuvant is dimethylsiloxane, dimethylsulfoxide, or a combination thereof.

46. A method for administering a local anesthetic agent to a patient comprising topically administering to the patient's body surface a composition comprising (a) a therapeutically effective amount of a local anesthetic agent and (b) a pharmaceutically acceptable, nonliposomal carrier comprised of a monohydric alcohol, a penetration enhancer, and a polymer selected from the group consisting of hydrophilic polymers, hydrophobic polymers and combinations thereof, wherein local anesthetic activity is provided within about thirty, minutes following topical administration.

47. The method of claim 46, wherein the local anesthetic activity is provided for at least 4 hours following topical administration.

48. The method of claim 46, wherein the local anesthetic activity is provided for at least 6 hours following topical administration.

49. The method of claim 46, wherein the monohydric alcohol is selected to volatilize following application of the composition to a localized region of a patient's body surface, thereby forming a film within the localized region.

50. A drug delivery system for topical administration of a local anesthetic agent, wherein the system is in the form of a laminated composite comprising:
(a) a drug reservoir layer containing a pharmaceutical composition of (i) a therapeutically effective amount of a local anesthetic agent, (ii) a monohydric alcohol, and (iii) an effective enhancing amount of a penetration enhancer; and
(b) a backing layer laminated to the drug reservoir layer that serves as the outer surface of the system following application to a patient's body surface.

51. The system of claim 50, wherein the drug reservoir comprises a polymeric matrix of a pharmaceutically acceptable bioadhesive material that defines the basal surface of the system and serves to affix the device to a body surface.

52. The system of claim 50, further including a layer of a pharmaceutically acceptable bioadhesive material that defines the basal surface of the system and serves to affix the system to a body surface.

53. The system of claim 50, wherein the drug reservoir is comprised of a sealed compartment containing the pharmaceutical composition in a liquid or gel formulation.

54. The system of claim 50, further including a removable release liner covering the basal surface of the system prior to application to the patient's body surface.

55. The system of claim 50, wherein the drug reservoir layer is water soluble.

56. A method for administering a local anesthetic agent to a patient, comprising applying the drug delivery system of claim 50 to a predetermined region of the patient's body surface.
